(19) United States
(12) Patent Application Publication
(10) Pub. No.: US 2024/0319165 A1
(43) Pub. Date: Sep. 26, 2024

(54) NANOPORE SYSTEMS AND METHODS OF FABRICATION

(71) Applicant: ILLUMINA, INC., San Diego, CA (US)

(72) Inventors: Xu LIU, San Diego, CA (US); Sharis MINASSIAN, San Diego, CA (US); Boyan BOYANOV, San Diego, CA (US); Rean Silke MUSA, San Diego, CA (US); Arvin EMADI, San Diego, CA (US)

(21) Appl. No.: 18/575,254

(22) PCT Filed: Nov. 9, 2022
(86) PCT No.: PCT/US2022/049414

§ 371 Date: Dec. 28, 2023

Related U.S. Application Data
(60) Provisional application No. 63/264,080, filed on Nov. 15, 2021.

(51) Int. Cl. G01N 33/487

(52) U.S. Cl. CPC G01N 33/48721

(57) ABSTRACT

Systems for sequencing biopolymers and methods of manufacturing the systems are disclosed. In one example, such a system may include an application specific integrated circuit (ASIC) layer, a post array layer beneath the AISC layer, and a nanopore layer above the ASIC layer. The ASIC layer is formed by building active circuitry on a front side of a semiconductor wafer and polishing a back side of the semiconductor wafer. The post array layer is formed by etching a front side of a support substrate and the post array layer provides mechanical support to the ASIC layer. The nanopore layer contains membrane and nanopores. The membrane inhibits passage of water-soluble molecules and the nanopores permit passage of water-soluble molecules. In some embodiments, the system may have short through-substrate vias extending through the ASIC layer. In some embodiments, wafer bonding processes may be used when fabricating the system.

### CROSS-REFERENCE TO RELATED APPLICATIONS

This application claims priority to U.S. Provisional Application No. 63/264,080, filed Nov. 15, 2021, the content of which is incorporated by reference in its entirety.

### FIELD

The present technology relates generally to devices for determining the sequence of a biopolymer, such as a polynucleotide or polypeptide, and more specifically to nanopore sequencing devices.

### BACKGROUND

Some polynucleotide sequencing techniques involve performing a large number of controlled reactions on support surfaces or within predefined reaction chambers. The controlled reactions may then be observed or detected, and subsequent analysis may help identify properties of the polynucleotide involved in the reaction. Examples of such sequencing techniques include next-generation sequencing or massive parallel sequencing involving sequencing-by-ligation, sequencing-by-synthesis, reversible terminator chemistry, or pyrosequencing approaches.

Some polynucleotide sequencing techniques utilize a nanopore, which can provide a path for an ionic electrical current. For example, as the polynucleotide traverses through the nanopore, it influences the ionic current through the nanopore. Each nucleotide, or series of nucleotides, that passes through the nanopore yields a characteristic electrical current. These characteristic electrical currents as a result of the traversing polynucleotide can be recorded to determine the sequence of the polynucleotide.

### SUMMARY

Provided in examples herein are devices for sequencing biopolymers, e.g., polynucleotides or proteins, and methods of manufacturing the devices. In one example, such a device has at least one nanopore and a fluidic channel (e.g., a vertical channel and/or a horizontal channel; the “horizontal” direction refers to a direction substantially parallel to the surface of a substrate, such as a glass or silicon wafer). When one or more monomers (e.g., nucleotides or amino acids) of the biopolymer are near or at the nanopore, the electrical resistance of the nanopore may vary in response to the identity of the one or more monomer units. The fluidic channel may have a fixed, or a substantially fixed electrical resistance. In some embodiments, a fluidic channel may be made with a small diameter in order to provide a large electrical resistance. In some embodiments, a horizontal channel may take a tortuous/serpentine/meandering path in order to achieve a long path length while keeping the footprint small. In some embodiments, such a device may include integrated electronics to take electrical measurements and/or to actively control in various parts of the device. Further details regarding example sequencing devices may be found in U.S. Provisional Patent Application No. 63/202,971 or International Application No. PCT/US2022/035837, entitled “DEVICE HAVING HORIZONTAL NANOCHANNEL FOR NANOPORE SEQUENCING”, the disclosures of which are incorporated herein by reference. The disclosed technology provides methods for manufacturing such sequencing devices without the need for etching long, expensive through-substrate vias (or through-silicon vias) in the device substrate (e.g., a silicon wafer) to achieve fluidic connections. Thus, the disclosed technology can improve the quality of the fabrication process and can provide a better device yield.

The systems, devices, kits, and methods disclosed herein each have several aspects, no single one of which is solely responsible for their desirable attributes. Without limiting the scope of the claims, some prominent features will now be discussed briefly. Numerous other examples are also contemplated, including examples that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

One example is a method of forming a bonded structure, the method comprising: providing a semiconductor wafer, wherein a front side of the semiconductor wafer includes active circuitry; thinning a back side of the semiconductor wafer; bonding a support substrate to the back side of the semiconductor wafer after said thinning the back side of the semiconductor wafer such that a front side of the support substrate is in contact with the back side of the semiconductor wafer; and etching the semiconductor wafer to form a plurality of fluidic channels, wherein each of the plurality of fluidic channels extends from the front side of the semiconductor wafer to the front side of the support substrate.

Another example is a method of forming a bonded structure, the method comprising: providing a semiconductor wafer, wherein a front side of the semiconductor wafer includes active circuitry; providing a support substrate; etching a front side of the support substrate to form a post array; thinning a back side of the semiconductor wafer; and bonding the support substrate to the back side of the semiconductor wafer after said thinning the back side of the semiconductor wafer such that the post array of the support substrate is in contact with the back side of the semiconductor wafer.

Still another example is a nanopore device comprising: an application specific integrated circuit (ASIC) layer, wherein the ASIC layer is formed by building active circuitry on a front side of a semiconductor wafer and polishing a back side of the semiconductor wafer; a post array layer beneath the ASIC layer, wherein the post array layer is formed by etching a front side of a support substrate and the post array layer provides mechanical support to the ASIC layer; and a nanopore layer above the ASIC layer, wherein the nanopore layer comprises membrane and a plurality of nanopores, wherein the membrane inhibits passage of water-soluble molecules and each of the plurality of nanopores permits passage of water-soluble molecules.

It is to be understood that any features of the device and/or of the array disclosed herein may be combined together in any desirable manner and/or configuration. Further, it is to be understood that any features of the method of using the device may be combined together in any desirable manner. Moreover, it is to be understood that any combination of features of this method and/or of the device and/or of the array may be used together, and/or may be combined with any of the examples disclosed herein. Still further, it is to be understood that any feature or combination of features of any of the devices and/or of the arrays and/or of any of the methods may be combined together in any desirable manner, and/or may be combined with any of the examples disclosed herein.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A illustrates a cross-sectional view of a nanopore device according to one embodiment of the disclosed technology.

FIG. 1B illustrates a cross-sectional view of a nanopore device according to another embodiment of the disclosed technology.

FIG. 2 is a top view illustrating a two-dimensional post array on a support substrate.

FIG. 3 is a flow diagram depicting a method for forming a bonded structure.

FIG. 4A is a cross-sectional view illustrating a semiconductor wafer for forming a nanopore device according to one embodiment of the disclosed technology.

FIG. 4B is a cross-sectional view illustrating a semiconductor wafer and a carrier substrate in a stage for forming a nanopore device according to one embodiment of the disclosed technology.

FIG. 4C and FIG. 4D are cross-sectional views illustrating a semiconductor wafer and a carrier substrate in different stages for forming a nanopore device according to one embodiment of the disclosed technology.

FIG. 4E is a cross-sectional view illustrating a semiconductor wafer and a carrier substrate in a stage for forming a nanopore device according to one embodiment of the disclosed technology.

FIG. 4F is a cross-sectional view illustrating a semiconductor wafer, a carrier substrate and a support substrate in a stage for forming a nanopore device according to one embodiment of the disclosed technology.

FIG. 4G is a cross-sectional view illustrating a semiconductor wafer and a support substrate in a stage for forming a nanopore device according to one embodiment of the disclosed technology.

FIG. 4H and FIG. 4I are cross-sectional and top views illustrating a semiconductor wafer and a support substrate in a stage for forming a nanopore device according to one embodiment of the disclosed technology.

FIG. 4J and FIG. 4K are cross-sectional and top views illustrating a semiconductor wafer and a support substrate in a stage for forming a nanopore device according to one embodiment of the disclosed technology.

FIG. 4L is a cross-sectional view illustrating a semiconductor wafer and a support substrate in a stage for forming a nanopore device according to one embodiment of the disclosed technology.

FIG. 4M is a cross-sectional view illustrating a semiconductor wafer and a support substrate in a stage for forming a nanopore device according to one embodiment of the disclosed technology.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, the terms “fluidically connecting,” “fluid communication,” “fluidically coupled,” and the like refer to two spatial regions being connected together such that a fluid (e.g., liquid or gas) may flow between the two spatial regions. For example, a cis well/wells may be fluidically connected to a trans well/wells by way of a middle well and/or a nanochannel, such that a fluid, e.g., at least a portion of an electrolyte, may flow between the connected wells.

As used herein, the term “ionic connection” and the like refer to two spatial regions being connected together such that certain species of ions may flow between the two spatial regions.

As used herein, the term “electric connection” and the like refer to two spatial regions being connected together such that electrons, holes, ions or other charge carriers may flow between the two spatial regions.

If an electrolyte flows between two connected wells, ions and electric currents may also flow between the connected wells. In some examples, two spatial regions may be in fluid/ionic/electric communication through first and second nanoscale openings, or through one or more valves, restrictors, or other fluidic components that are to control or regulate a flow of fluid, ions or electric current through a system.

As used herein, the term “operably connected” refers to a configuration of elements, wherein an action or reaction of one element affects another element, but in a manner that preserves each element's functionality.

As used herein, the term “membrane” refers to a non-permeable or semi-permeable barrier or other sheet that separates two liquid/gel chambers (e.g., a cis well and a fluidic cavity or reservoir) which can contain the same compositions or different compositions therein. The permeability of the membrane to any given species depends upon the nature of the membrane. In some examples, the membrane may be non-permeable to ions, to electric current, and/or to fluids. For example, a lipid membrane may be impermeable to ions (i.e., does not allow any ion transport therethrough), but may be at least partially permeable to water (e.g., water diffusivity ranges from about 40 μm/s to about 100 μm/s). For another example, a synthetic/solid-state membrane, one example of which is silicon nitride, may be impermeable to ions, electric charge, and fluids (i.e., the diffusion of all of these species is zero). Any membrane may be used in accordance with the present disclosure, as long as the membrane can include a transmembrane nanoscale opening and can maintain a potential difference across the membrane. The membrane may be a monolayer or a multilayer membrane. A multilayer membrane includes two or more layers, each of which is a non-permeable or semi-permeable material.

The membrane may be formed of materials of biological or non-biological origin. A material that is of biological origin refers to material derived from or isolated from a biological environment such as an organism or cell, or a synthetically manufactured version of a biologically available structure (e.g., a biomimetic material).

An example membrane that is made from the material of biological origin includes a monolayer formed by a bolalipid. Another example membrane that is made from the material of biological origin includes a lipid bilayer. Suitable lipid bilayers include, for example, a membrane of a cell, a membrane of an organelle, a liposome, a planar lipid bilayer, and a supported lipid bilayer. A lipid bilayer can be formed, for example, from two opposing layers of phospholipids, which are arranged such that their hydrophobic tail groups face towards each other to form a hydrophobic interior, whereas the hydrophilic head groups of the lipids face outwards towards the aqueous environment on each side of the bilayer. Lipid bilayers also can be formed, for example, by a method in which a lipid monolayer is carried on an aqueous solution/air interface past either side of an aperture that is substantially perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has at least partially evaporated, the solution/air interfaces on either side of the aperture are physically moved up and down past the aperture until a bilayer is formed. Other suitable methods of bilayer formation include tip-dipping, painting bilayers, and patch-clamping of liposome bilayers. Any other methods for obtaining or generating lipid bilayers may also be used.

A material that is not of biological origin may also be used as the membrane. Some of these materials are solid-state materials and can form a solid-state membrane, and others of these materials can form a thin liquid film or membrane. The solid-state membrane can be a monolayer, such as a coating or film on a supporting substrate (i.e., a solid support), or a freestanding element. The solid-state membrane can also be a composite of multilayered materials in a sandwich configuration. Any material not of biological origin may be used, as long as the resulting membrane can include a transmembrane nanoscale opening and can maintain a potential difference across the membrane. The membranes may include organic materials, inorganic materials, or both. Examples of suitable solid-state materials include, for example, microelectronic materials, insulating materials (e.g., silicon nitride (Si₃N₄), aluminum oxide (Al₂O₃), hafnium oxide (HfO₂), tantalum pentoxide (Ta₂O₅), silicon oxide (SiO₂), etc.), some organic and inorganic polymers (e.g., polyamide, plastics, such as polytetrafluoroethylene (PTFE), or elastomers, such as two-component addition-cure silicone rubber), and glasses. In addition, the solid-state membrane can be made from a monolayer of graphene, which is an atomically thin sheet of carbon atoms densely packed into a two-dimensional honeycomb lattice, a multilayer of graphene, or one or more layers of graphene mixed with one or more layers of other solid-state materials. A graphene-containing solid-state membrane can include at least one graphene layer that is a graphene nanoribbon or graphene nanogap, which can be used as an electrical sensor to characterize the target polynucleotide. It is to be understood that the solid-state membrane can be made by any suitable method, for example, chemical vapor deposition (CVD). In an example, a graphene membrane can be prepared through either CVD or exfoliation from graphite. Examples of suitable thin liquid film materials that may be used include diblock copolymers or triblock copolymers, such as amphiphilic PMOXA-PDMS-PMOXA ABA triblock copolymers.

As used herein, the term “nanopore” is intended to mean a hollow structure discrete from, or defined in, and extending across the membrane. The nanopore permits ions, electric current, and/or fluids to cross from one side of the membrane to the other side of the membrane. For example, a membrane that inhibits the passage of ions or water-soluble molecules can include a nanopore structure that extends across the membrane to permit the passage (through a nanoscale opening extending through the nanopore structure) of the ions or water-soluble molecules from one side of the membrane to the other side of the membrane. The diameter of the nanoscale opening extending through the nanopore structure can vary along its length (i.e., from one side of the membrane to the other side of the membrane), but at any point is on the nanoscale (i.e., from about 1 nm to about 100 nm, or to less than 1000 nm). Examples of the nanopore include, for example, biological nanopores, solid-state nanopores, and biological and solid-state hybrid nanopores.

As used herein, the term “diameter” is intended to mean a longest straight line inscribable in a cross-section of a nanoscale opening through a centroid of the cross-section of the nanoscale opening. It is to be understood that the nanoscale opening may or may not have a circular or substantially circular cross-section. Further, the cross-section may be regularly or irregularly shaped.

As used herein, the term “biological nanopore” is intended to mean a nanopore whose structure portion is made from materials of biological origin. Biological origin refers to a material derived from or isolated from a biological environment such as an organism or cell, or a synthetically manufactured version of a biologically available structure. Biological nanopores include, for example, polypeptide nanopores and polynucleotide nanopores.

As used herein, the term “polypeptide nanopore” is intended to mean a protein/polypeptide that extends across the membrane, and permits ions, electric current, biopolymers such as DNA or peptides, or other molecules of appropriate dimension and charge, and/or fluids to flow therethrough from one side of the membrane to the other side of the membrane. A polypeptide nanopore can be a monomer, a homopolymer, or a heteropolymer. Structures of polypeptide nanopores include, for example, an α-helix bundle nanopore and a β-barrel nanopore. Example polypeptide nanopores include α-hemolysin, Mycobacterium smegmatis porin A (MspA), gramicidin A, maltoporin, OmpF, OmpC, PhoE, Tsx, F-pilus, aerolysin, etc. The protein α-hemolysin is found naturally in cell membranes, where it acts as a pore for ions or molecules to be transported in and out of cells. Mycobacterium smegmatis porin A (MspA) is a membrane porin produced by Mycobacteria, which allows hydrophilic molecules to enter the bacterium. MspA forms a tightly interconnected octamer and transmembrane beta-barrel that resembles a goblet and contains a central pore.

A polypeptide nanopore can be synthetic. A synthetic polypeptide nanopore includes a protein-like amino acid sequence that does not occur in nature. The protein-like amino acid sequence may include some of the amino acids that are known to exist but do not form the basis of proteins (i.e., non-proteinogenic amino acids). The protein-like amino acid sequence may be artificially synthesized rather than expressed in an organism and then purified/isolated.

As used herein, the term “polynucleotide nanopore” is intended to include a polynucleotide that extends across the membrane, and permits ions, electric current, and/or fluids to flow from one side of the membrane to the other side of the membrane. A polynucleotide pore can include, for example, a polynucleotide origami (e.g., nanoscale folding of DNA to create the nanopore).

Also as used herein, the term “solid-state nanopore” is intended to mean a nanopore whose structure portion is defined by a solid-state membrane and includes materials of non-biological origin (i.e., not of biological origin). A solid-state nanopore can be formed of an inorganic or organic material. Solid-state nanopores include, for example, silicon nitride nanopores, silicon dioxide nanopores, and graphene nanopores.

The nanopores disclosed herein may be hybrid nanopores. A “hybrid nanopore” refers to a nanopore including materials of both biological and non-biological origins. An example of a hybrid nanopore includes a polypeptide-solid-state hybrid nanopore and a polynucleotide-solid-state nanopore.

In some embodiments, the nanopore may comprise a solid-state material, such as silicon nitride, modified silicon nitride, silicon, silicon oxide, or graphene, or a combination thereof. In some embodiments, the nanopore is a protein that forms a tunnel upon insertion into a bilayer, membrane, thin film, or solid-state aperture. In some embodiments, the nanopore is comprised in a lipid bilayer. In some embodiments, the nanopore is comprised in an artificial membrane comprising a mycolic acid. The nanopore may be a Mycobacterium smegmatis porin (Msp) having a vestibule and a constriction zone that define the tunnel. The Msp porin may be a mutant MspA porin. In some embodiments, amino acids at positions 90, 91, and 93 of the mutant MspA porin are each substituted with asparagine. Some embodiments may comprise altering the translocation velocity or sequencing sensitivity by removing, adding, or replacing at least one amino acid of an Msp porin. A “mutant MspA porin” is a multimer complex that has at least or at most 70, 75, 80, 85, 90, 95, 98, or 99 percent or more identity, or any range derivable therein, but less than 100%, to its corresponding wild-type MspA porin and retains tunnel-forming capability. A mutant MspA porin may be recombinant protein. Optionally, a mutant MspA porin is one having a mutation in the constriction zone or the vestibule of a wild-type MspA porin. Optionally, a mutation may occur in the rim or the outside of the periplasmic loops of a wild-type MspA porin. A mutant MspA porin may be employed in any embodiment described herein.

A “vestibule” refers to the cone-shaped portion of the interior of an Msp porin whose diameter generally decreases from one end to the other along a central axis, where the narrowest portion of the vestibule is connected to the constriction zone. A vestibule may also be referred to as a “goblet.” The vestibule and the constriction zone together define the tunnel of an Msp porin. A “constriction zone” or the “readhead” refers to the narrowest portion of the tunnel of an Msp porin, in terms of diameter, that is connected to the vestibule. The length of the constriction zone may range from about 0.3 nm to about 2 nm. Optionally, the length is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. The diameter of the constriction zone may range from about 0.3 nm to about 2 nm. Optionally, the diameter is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. A “tunnel” refers to the central, empty portion of an Msp porin that is defined by the vestibule and the constriction zone, through which a gas, liquid, ion, or analyte may pass. A tunnel is an example of an opening of a nanopore.

Various conditions such as light and the liquid medium that contacts a nanopore, including its pH, buffer composition, detergent composition, and temperature, may affect the behavior of the nanopore, particularly with respect to its conductance through the tunnel as well as the movement of an analyte with respect to the tunnel, either temporarily or permanently.

In some embodiments, the disclosed system for nanopore sequencing comprises an Msp porin having a vestibule and a constriction zone that define a tunnel, wherein the tunnel is positioned between a first liquid medium and a second liquid medium, wherein at least one liquid medium comprises an analyte polynucleotide, and wherein the system is operative to detect a property of the analyte. The system may be operative to detect a property of any analyte comprising subjecting an Msp porin to an electric field such that the analyte interacts with the Msp porin. The system may be operative to detect a property of the analyte comprising subjecting the Msp porin to an electric field such that the analyte electrophoretically translocates through the tunnel of the Msp porin. In some embodiments, the system comprises an Msp porin having a vestibule and a constriction zone that define a tunnel, wherein the tunnel is positioned in a lipid bilayer between a first liquid medium and a second liquid medium, and wherein the only point of liquid communication between the first and second liquid media occurs in the tunnel. Moreover, any Msp porin described herein may be comprised in any system described herein.

The system may further comprise one or more temperature regulating devices in communication with the fluid or electrolyte. The system described herein may be operative to translocate an analyte through an Msp porin tunnel either electrophoretically or otherwise.

As used herein, the term “nanopore sequencer” refers to any of the devices disclosed herein that can be used for nanopore sequencing. In the examples disclosed herein, during nanopore sequencing, the nanopore is immersed in example(s) of the electrolyte disclosed herein and a potential difference is applied across the membrane. In an example, the potential difference is an electric potential difference or an electrochemical potential difference. An electrical potential difference can be imposed across the membrane via a voltage source that injects or administers current to at least one of the ions of the electrolyte contained in the cis well or one or more of the trans wells. An electrochemical potential difference can be established by a difference in ionic composition of the cis and trans wells in combination with an electrical potential. The different ionic composition can be, for example, different ions in each well or different concentrations of the same ions in each well.

The application of the potential difference across a nanopore may force the translocation of a nucleic acid through the nanopore. One or more signals are generated that correspond to the translocation of the nucleotide through the nanopore. Accordingly, as a target polynucleotide, or as a mononucleotide or a probe derived from the target polynucleotide or mononucleotide, transits through the nanopore, the current across the membrane changes due to base-dependent (or probe dependent) blockage of the constriction, for example. The signal from that change in current can be measured using any of a variety of methods. Each signal is unique to the species of nucleotide(s) (or probe) in the nanopore, such that the resultant signal can be used to determine a characteristic of the polynucleotide. For example, the identity of one or more species of nucleotide(s) (or probe) that produces a characteristic signal can be determined.

As used herein, a “reporter” is composed of one or more reporter elements. Reporters include what are known as “tags” and “labels.” Reporters serve to parse the genetic information of the target nucleic acid. “Encode” or “parse” are verbs referring to transferring from one format to another, and refers to transferring the genetic information of target template base sequence into an arrangement of reporters.

As used herein, a “peptide” refers to two or more amino acids joined together by an amide bond (that is, a “peptide bond”). Peptides comprise up to or include 50 amino acids. Peptides may be linear or cyclic. Peptides may be α, β, γ, δ, or higher, or mixed. Peptides may comprise any mixture of amino acids as defined herein, such as comprising any combination of D, L, α, β, γ, δ, or higher amino acids.

As used herein, a “protein” refers to an amino acid sequence having 51 or more amino acids.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides are natural nucleotides, but it is to be further understood that non-natural nucleotides, modified nucleotides or analogs of the aforementioned nucleotides can also be used.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing. The term “nucleic acid” may be used interchangeably with “polynucleotide” to refer to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

For example, a template polynucleotide chain may be any sample that is to be sequenced, and may be composed of DNA, RNA, or analogs thereof (e.g., peptide nucleic acids). The source of the template (or target) polynucleotide chain can be genomic DNA, messenger RNA, or other nucleic acids from native sources. In some cases, the template polynucleotide chain that is derived from such sources can be amplified prior to use. Any of a variety of known amplification techniques can be used including, but not limited to, polymerase chain reaction (PCR), rolling circle amplification (RCA), multiple displacement amplification (MDA), or random primer amplification (RPA). It is to be understood that amplification of the template polynucleotide chain prior to use is optional. As such, the template polynucleotide chain will not be amplified prior to use in some examples. Template/target polynucleotide chains can optionally be derived from synthetic libraries. Synthetic nucleic acids can have native DNA or RNA compositions or can be analogs thereof.

Biological samples from which the template polynucleotide chain can be derived include, for example, those from a mammal, such as a rodent, mouse, rat, rabbit, guinea pig, ungulate, horse, sheep, pig, goat, cow, cat, dog, primate, human or non-human primate; a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii Takifugu rubripes, yeast, Saccharamoyces cerevisiae or Schizosaccharomyces pombe; or a Plasmodium falciparum. Template polynucleotide chains can also be derived from prokaryotes such as a bacterium, Escherichia coli, staphylococci or Mycoplasma pneumoniae; an archae; a virus such as Hepatitis C virus, ebola virus or human immunodeficiency virus; or a viroid. Template polynucleotide chains can be derived from a homogeneous culture or population of the above organisms or alternatively from a collection of several different organisms, for example, in a community or ecosystem.

Moreover, template polynucleotide chains may not be derived from natural sources, but rather can be synthesized using known techniques. For example, gene expression probes or genotyping probes can be synthesized and used in the examples set forth herein.

In some examples, template polynucleotide chains can be obtained as fragments of one or more larger nucleic acids. Fragmentation can be carried out using any of a variety of techniques known in the art including, for example, nebulization, sonication, chemical cleavage, enzymatic cleavage, or physical shearing. Fragmentation may also result from use of a particular amplification technique that produces amplicons by copying only a portion of a larger nucleic acid chain. For example, PCR amplification produces fragments having a size defined by the length of the nucleotide sequence on the original template that is between the locations where flanking primers hybridize during amplification. The length of the template polynucleotide chain may be in terms of the number of nucleotides or in terms of a metric length (e.g., nanometers).

A population of template/target polynucleotide chains, or amplicons thereof, can have an average strand length that is desired or appropriate for a particular sequencing device. For example, the average strand length can be less than about 100,000 nucleotides, about 50,000 nucleotides, about 10,000 nucleotides, about 5,000 nucleotides, about 1,000 nucleotides, about 500 nucleotides, about 100 nucleotides, or about 50 nucleotides. Alternatively or additionally, the average strand length can be greater than about 10 nucleotides, about 50 nucleotides, about 100 nucleotides, about 500 nucleotides, about 1,000 nucleotides, about 5,000 nucleotides, about 10,000 nucleotides, about 50,000 nucleotides, or about 100,000 nucleotides. Alternatively or additionally, the average strand length can be greater than about 10 kilo nucleotides, about 50 kilo nucleotides, about 100 kilo nucleotides, about 500 kilo nucleotides, about 1,000 kilo nucleotides, about 5,000 kilo nucleotides, about 10,000 kilo nucleotides, about 50,000 kilo nucleotides, or about 100,000 kilo nucleotides. Alternatively or additionally, the average strand length can be greater than about 10 mega nucleotides, about 50 mega nucleotides, about 100 mega nucleotides, about 500 mega nucleotides, about 1,000 mega nucleotides, about 5,000 mega nucleotides, about 10,000 mega nucleotides, about 50,000 mega nucleotides, or about 100,000 mega nucleotides. The average strand length for a population of target polynucleotide chains, or amplicons thereof, can be in a range between a maximum and minimum value set forth above.

In some cases, a population of template/target polynucleotide chains can be produced under conditions or otherwise configured to have a maximum length for its members. For example, the maximum length for the members can be less than about 100,000 nucleotides, about 50,000 nucleotides, about 10,000 nucleotides, about 5,000 nucleotides, about 1,000 nucleotides, about 500 nucleotides, about 100 nucleotides or about 50 nucleotides. For example, the maximum length for the members can be less than about 100,000 kilo nucleotides, about 50,000 kilo nucleotides, about 10,000 kilo nucleotides, about 5,000 kilo nucleotides, about 1,000 kilo nucleotides, about 500 kilo nucleotides, about 100 kilo nucleotides or about 50 kilo nucleotides. For example, the maximum length for the members can be less than about 100,000 mega nucleotides, about 50,000 mega nucleotides, about 10,000 mega nucleotides, about 5,000 mega nucleotides, about 1,000 mega nucleotides, about 500 mega nucleotides, about 100 mega nucleotides or about 50 mega nucleotides. Alternatively or additionally, a population of template polynucleotide chains, or amplicons thereof, can be produced under conditions or otherwise configured to have a minimum length for its members. For example, the minimum length for the members can be more than about 10 nucleotides, about 50 nucleotides, about 100 nucleotides, about 500 nucleotides, about 1,000 nucleotides, about 5,000 nucleotides, about 10,000 nucleotides, about 50,000 nucleotides, or about 100,000 nucleotides. For example, the minimum length for the members can be more than about 10 kilo nucleotides, about 50 kilo nucleotides, about 100 kilo nucleotides, about 500 kilo nucleotides, about 1,000 kilo nucleotides, about 5,000 kilo nucleotides, about 10,000 kilo nucleotides, about 50,000 kilo nucleotides, or about 100,000 kilo nucleotides. For example, the minimum length for the members can be more than about 10 mega nucleotides, about 50 mega nucleotides, about 100 mega nucleotides, about 500 mega nucleotides, about 1,000 mega nucleotides, about 5,000 mega nucleotides, about 10,000 mega nucleotides, about 50,000 mega nucleotides, or about 100,000 mega nucleotides. The maximum and minimum strand length for template polynucleotide chains in a population can be in a range between a maximum and minimum value set forth above.

As used herein, the term “signal” is intended to mean an indicator that represents information. Signals include, for example, an electrical signal and an optical signal. The term “electrical signal” refers to an indicator of an electrical quality that represents information. The indicator can be, for example, current, voltage, tunneling, resistance, potential, voltage, conductance, or a transverse electrical effect (and any time-derivatives or transients of theses). An “electronic current” or “electric current” refers to a flow of electric charge. In an example, an electrical signal may be an electric current passing through a nanopore, and the electric current may flow when an electric potential difference is applied across the nanopore.

The term “substrate” refers to a rigid, solid support that is insoluble in aqueous liquid and is incapable of passing a liquid absent an aperture, port, or other like liquid conduit. In the examples disclosed herein, the substrate may have wells or chambers defined therein. Examples of suitable substrates include glass and modified or functionalized glass, plastics (including acrylics, polystyrene and copolymers of styrene and other materials, polypropylene, polyethylene, polybutylene, polyurethanes, polytetrafluoroethylene (PTFE) (such as TEFLON® from Chemours), cyclic olefins/cyclo-olefin polymers (COP) (such as ZEONOR® from Zeon), polyimides, etc.), nylon, ceramics, silica or silica-based materials, silicon and modified silicon, carbon, metals, inorganic glasses, and optical fiber bundles.

The terms top, bottom, lower, upper, on, etc. are used herein to describe the device/nanopore sequencer and/or the various components of the device. It is to be understood that these directional terms are not meant to imply a specific orientation, but are used to designate relative orientation between components. The use of directional terms should not be interpreted to limit the examples disclosed herein to any specific orientation(s). As used herein, the terms “upper”, “lower”, “vertical”, “horizontal” and the like are meant to indicate relative orientation.

As used herein, “cis” refers to the side of a nanopore opening through which an analyte or modified analyte enters the opening or across the face of which the analyte or modified analyte moves.

As used herein, “trans” refers to the side of a nanopore opening through which an analyte or modified analyte (or fragments thereof) exits the opening or across the face of which the analyte or modified analyte does not move.

As used herein, by “translocation,” it is meant that an analyte (e.g., DNA) enters one side of an opening of a nanopore and move to and out of the other side of the opening. It is contemplated that any embodiment herein comprising translocation may refer to electrophoretic translocation or non-electrophoretic translocation, unless specifically noted. An electric field may move an analyte (e.g., a polynucleotide) or modified analyte. By “interacts,” it is meant that the analyte (e.g., DNA) or modified analyte moves into and, optionally, through the opening, where “through the opening” (or “translocates”) means to enter one side of the opening and move to and out of the other side of the opening. Optionally, methods that do not employ electrophoretic translocation are contemplated. In some embodiments, physical pressure causes a modified analyte to interact with, enter, or translocate (after alteration) through the opening. In some embodiments, a magnetic bead is attached to an analyte or modified analyte on the trans side, and magnetic force causes the modified analyte to interact with, enter, or translocate (after alteration) through the opening. Other methods for translocation include but not limited to gravity, osmotic forces, temperature, and other physical forces such as centripetal force.

As used herein, the terms “well”, “cavity”, “reservoir” and “chamber” are used synonymously, and refer to a discrete feature defined in the device that can contain a fluid (e.g., liquid, gel, gas). A cis well is a chamber that contains or is partially defined by a cis electrode, and is also fluidically connected to a middle well where measurements occur (for example, by a FET, or by a metal electrode connected to an amplifier, a data acquisition device, or other signal conditioning elements such as analog filters, buffers, gain amplifiers, ADCs, etc.). The middle well in turn is fluidically connected to a trans well/chamber, in some examples. Examples of an array of the present device may have one cis well, for example one global cis chamber/reservoir, or multiple cis wells. The trans well is a single chamber that contains or is partially defined by its own trans electrode, and is also fluidically connected to a cis well. In examples including multiple trans wells, each trans well is electrically isolated from each other trans well. Further, it is to be understood that the cross-section of a well taken parallel to a surface of a substrate at least partially defining the well can be curved, square, polygonal, hyperbolic, conical, angular, etc. As used herein, “field-effect transistors” or “FETs” typically include doped source/drain regions that are formed of a semiconductor material, e.g., silicon, germanium, gallium arsenide, silicon carbide, etc., and are separated by a channel region. A n-FET is a FET having an n-channel in which the current carriers are electrons. A p-FET is a FET having a p-channel in which the current carriers are holes. Source/drain regions of a n-FET device may include a different material than source/drain regions of a p-FET device. In some examples, the source/drain regions or the channel may not be doped. Doped regions may be formed by adding dopant atoms to an intrinsic semiconductor. This changes the electron and hole carrier concentrations of the intrinsic semiconductor at thermal equilibrium. A doped region may be p-type or n-type. As used herein, “p-type” refers to the addition of impurities to an intrinsic semiconductor that creates a deficiency of valence electrons. For silicon, example p-type dopants, i.e., impurities, include but are not limited to boron, aluminum, gallium, and indium. As used herein, “n-type” refers to the addition of impurities that contribute free electrons to an intrinsic semiconductor. For silicon, example n-type dopants, i.e., impurities, include but are not limited to, antimony, arsenic, and phosphorus. The dopant(s) may be introduced by ion implantation or plasma doping.

For example, in an integrated circuit having a plurality of metal oxide semiconductor field effect transistors (MOSFETs), each MOSFET has a source and a drain that are formed in an active region of a semiconductor layer by implanting n-type or p-type impurities in the layer of semiconductor material. Disposed between the source and the drain is a channel (or body) region. Disposed above the body region is a gate electrode. The gate electrode and the body are spaced apart by a gate dielectric (gate oxide) layer. The channel region connects the source and the drain, and electrical current flows through the channel region from the source to the drain. The electrical current flow is induced in the channel region by a voltage applied at the gate electrode.

In some embodiments, the channel of a FET sensor located between the source and drain may be covered by a relatively thin layer of a gate oxide, for example a thermally grown silicon dioxide layer. Alternatively, a thin layer of an insulator may be formed of high-K dielectrics, such as HfO₂, Al₂O₃, silicon nitroxides, Si₃N₄, TiO₂, Ta₂O₅, Y₂O₃, La₂O₃, ZrO₂, ZrSiO₄, barium strontium titanate, lead zirconate titanate, ZrSiₓO_{y}, or ZrAlₓO_{y}. The layer of gate oxide may be about 10 nm in thickness, or in other examples, less than about 9, about 8, about 7, about 6, about 5, about 4, about 3, about 2, or about 1 nm in thickness.

The aspects and examples set forth herein and recited in the claims can be understood in view of the above definitions.

### Nanopore Sequencing

Polynucleotides may be sequenced using a nanopore unit cell, or a nanopore sensor, based on electrical responses. In some embodiments, such unit cell may include a nanopore, a flow chamber containing a liquid, one or more electrodes, and an electronic circuit for measurement. In some cases, the nanopore may be a solid-state nanopore. In some cases, the nanopore may be a solid-state nanopore directly formed as a nanoscale opening in a membrane (e.g., silicon based, graphene, or polymer membrane). A polynucleotide may be dissolved in the liquid, e.g., an electrolyte. In some embodiments, application of a voltage via the one or more electrodes results in a driving force and/or a change in the electrical conditions that are suitable for driving translocation of the polynucleotide through the nanopore, for example from the “cis” side to the “trans” side. As the polynucleotide translocates through the nanopore, the polynucleotide may modulate the electrical properties of the nanopore, such that the nucleobase sequence of the polynucleotide can be identified. For example, the electrical current through the nanopore or the electrical resistance at the nanopore may be a function of the identity of the nucleobase of the polynucleotide at or near the nanopore.

In some cases, the nanopore may be a biological nanopore formed of peptides or polynucleotides and inserted in a lipid bilayer or a polymer membrane, e.g., a synthetic polymeric membrane. In an example, a protein nanopore is deposited in a lipid bilayer. A single-stranded DNA may pass, from the “cis” side, through the nanopore, to the “trans” side. Applying a voltage across the “cis” side to the “trans” side results in an ionic current through the nanopore. When a nucleotide of the DNA is in or near the nanopore, it may result in a unique ionic current blockade at the nanopore, and therefore a unique nanopore resistance depending on the identity of the nucleotide. By measuring the ionic current or the nanopore resistance, the nucleotide at or near the nanopore can be identified. It is to be understood that the disclosed nanopore sequencing technologies can be used in combination with any of the aforementioned scenarios.

In various embodiments, determining a signal level of the nanopore includes, alone or in combination, determining the signal level by measuring electrical characteristics of the cis/trans nanopore cell, such as determining the ionic current through the nanopore, determining the voltage potential at a specified area or component of the cis/trans nanopore cell, determining the electrical impedance at a specified area or component of the cis/trans nanopore cell, or determining the conductivity/resistance of the nanopore membrane.

In other embodiments, the DNA may not pass through the nanopore. A unique tag or label for a nucleotide in the DNA may pass through the nanopore. In one example, a tag or label of the nucleotide may be a particular sequence combination of nucleotides. When the tag or label is in or near the nanopore, it may result in a unique ionic current blockade at the nanopore, and therefore a unique nanopore resistance depending on the identity of the molecule of interest. By measuring the ionic current or the nanopore resistance, the tag or label at or near the nanopore, and therefore the corresponding nucleotide, can be identified. In some embodiments, sequencing of a target polynucleotide may involve nanopore sensing of (1) a single-stranded portion of the target polynucleotide; (2) a nucleic acid duplex of a portion of the target polynucleotide; (3) a label or tag that can be tethered or untethered to the target polynucleotide; or any combination thereof. It is to be understood that the disclosed nanopore sequencing technologies can be used in combination with any of the aforementioned scenarios.

In some embodiments, the nanopore resistor may be connected in series with another resistor (e.g. the resistance of a fluidic channel). As the nanopore resistance varies because of the identity of the molecule passed through the nanopore, voltage on the point between the two resistors changes. Such change can be measured by active electronic measurement circuits to identify the molecule. As such, the two resistors form a voltage divider. The voltage on the dividing point (i.e. the connecting point between the two resistors) can be measured by active electronic measurement circuits to identify the molecule that passes through the nanopore. Further details regarding example sequencing devices may be found in U.S. Provisional Patent Application No. 63/047,743 or No. 63/200,868 or International Application No. PCT/US2021/038125, entitled “DEVICES WITH FIELD EFFECT TRANSISTORS”, the disclosures of which are incorporated herein by reference.

In some embodiments, multiple nanopore sensors may be arranged in an array, each nanopore sensor corresponding to a sequencing channel for identifying the nucleobase sequence of a target polynucleotide. In some embodiments, each nanopore sensor may be independently accessed or controlled by a logic circuit. For example, nanopore sensors may be controlled by a controller implemented in hardware, software, or both, such as CPUs, GPUs, FPGA, microcontroller, or microprocessors. In some embodiments, a nanopore array is formed of a two-dimensional or a three-dimensional arrangement of nanopore sensors/nanopore unit cells. Each nanopore sensor/nanopore unit cell may be used to sequence a polynucleotide molecule. A nanopore array may therefore be used to sequence a plurality of polynucleotide molecules in parallel. In some cases, a nanopore array may be used to sequence a plurality of polynucleotide molecules substantially simultaneously. It is to be understood that the disclosed nanopore sequencing technologies can be used in combination with any of the aforementioned scenarios.

The array may have any suitable number of nanopore unit cells. In some instances, the array comprises about 200, about 400, about 600, about 800, about 1000, about 1500, about 2000, about 3000, about 4000, about 5000, about 10000, about 15000, about 20000, about 40000, about 60000, about 80000, about 100000, about 200000, about 400000, about 600000, about 800000, about 1000000, about 10000000 or more nanopore unit cells. In some instances, the array comprises at least 200, at least 400, at least 600, at least 800, at least 1000, at least 1500, at least 2000, at least 3000, at least 4000, at least 5000, at least 10000, at least 15000, at least 20000, at least 40000, at least 60000, at least 80000, at least 100000, at least 200000, at least 400000, at least 600000, at least 800000, at least 1000000 or at least 10000000 nanopore unit cells. In some cases, the array can include individually addressable nanopore unit cells at a density of at least about 500, 600, 700, 800, 900, 1000, 10,000, 100,000 or 1,000,000 unit cells per mm².

To independently control or address each unit cell, in some embodiments, each of the nanopore unit cells in a nanopore array may have its own trans electrode but may share a common cis electrode. In some embodiments, each of the nanopore unit cells in a nanopore array may have its own cis electrode but may share a common trans electrode. In some embodiments, each of the nanopore unit cells in a nanopore array may have its own cis electrode and trans electrode. In some embodiments, each of the nanopore unit cells in a nanopore array may share a common cis electrode and a common trans electrode.

### Overview of the Disclosed Technology

In some embodiments, a nanopore sequencing system may include an array of nanopores, respective fluidic wells, respective fluidic channels/nanochannels, and a thin layer of active electronic devices used for signal detection. In one aspect, the disclosed technology provides a fabrication process for a nanopore sequencing system. In another aspect, the disclosed technology provides a product of such a fabrication process. In some embodiments, the fabrication process may provide a way for connecting different fluidic wells of the system, for example fluidic wells located at the frontside and the backside of the thin layer of active electronic devices (e.g., a semiconductor layer comprising application-specific integrated circuit, or ASIC, sensors). In some embodiments, the system may have short through-substrate vias (e.g., about 1-about 10 microns in length) extending through the thin layer of active electronic devices. In some embodiments, the system may further include an array (e.g., a two-dimensional, or 2D, array) of post structures which serve to create a fluidic reservoir connecting to the fluidic tunnels. The array of post or pillar structures may also serve to mechanically support the thin layer of active electronic devices. In some embodiments, wafer bonding processes may be used when fabricating the system. For example, the fabrication process may include transferring and temporarily bonding a wafer hosting active electronic devices to another wafer providing structural support, thinning down the wafer hosting active electronic devices from its backside, bonding yet another wafer to the backside of the wafer hosting active electronic devices to create fluidic features, and debonding the wafer providing structural support. In some cases, electrolyte depletion is a critical issue in nanopore sensing systems. A system having a large common cis well and a large common trans well, such as a dual pore implementation described in U.S. Provisional Patent Application No. 63/202,971 or 63/261,937 or International Patent Application No. PCT/US2022/035837 or PCT/US2021/038125, the disclosure of each of which being incorporated herein by reference, may address this issue. However, as the frontside and backside of an active electronics layer of such a system need to be fluidically connected in some examples, certain designs of the dual pore implementation may include a long (e.g., more than 20 microns in length) through-silicon via/through-substrate via (TSV) structure which is expensive or difficult to manufacture/difficult to control and which limits the density of nanopore sensor subunits that can be achieved in a given substrate.

In some embodiments, using only short TSVs through thin substrates (e.g., 2-10 microns in thickness), the disclosed technology may prevent the need for etching long, expensive TSVs to achieve fluidic connections in the system. Thus, the disclose technology may improve the quality of the fabrication process and provide a better manufacturing yield. Further, the disclose technology may provide manufacturing processes which cost less and can achieve higher nanopore sensor subunit density in the substrate. In addition, the low aspect ratio of the short TSVs connecting the frontside and backside of the active electronics layer allows the TSVs to be easier to wet by electrolytes when the system is being prepared for sequencing operations.

### Nanopore System

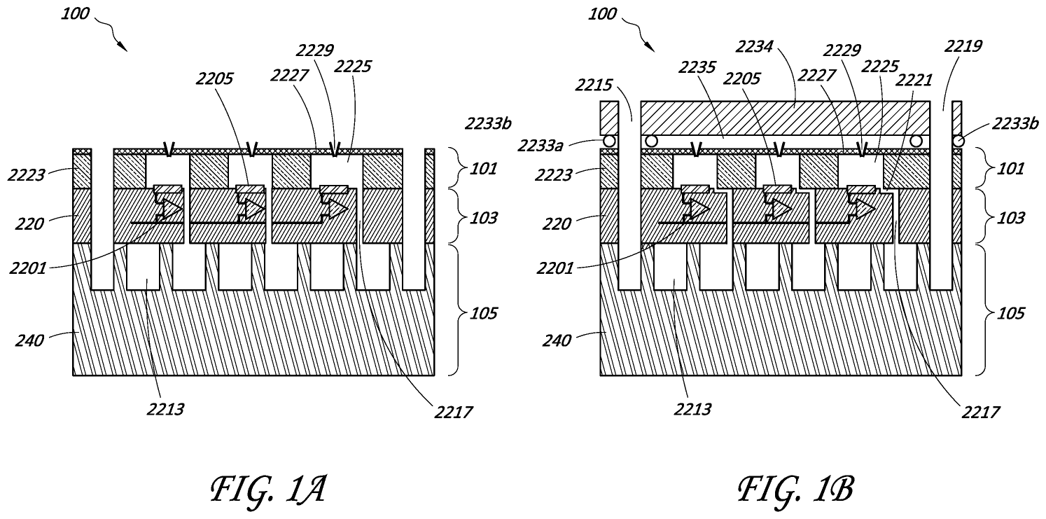

Provided herein is a nanopore system that exhibits improved voltage divider design. The nanopore system utilizes short through-substrate vias (TSVs) as fluidic channels, which simplifies the TSV fabrication and enables high nanopore pixel density. FIG. 1A illustrates a cross-sectional view of a nanopore device 100 according to one embodiment. The nanopore device 100 may be a flow cell that includes an application specific integrated circuit (ASIC) layer 103 between a nanopore layer 101 and a post array layer 105. The nanopore layer 101 is attached to a front side of the ASIC layer 103, and the post array layer 105 is attached to the back side of the ASIC layer 103.

The ASIC layer 103 includes active circuitry 2201 (e.g., semiconductor circuits with transistor amplifiers) built into the front side of a semiconductor wafer 220. Each active circuitry unit has a conductive pad 2205, such as a metal, metal compounds, and other conductive materials, on the front side of the ASIC layer 103. The conductive pads 2205 may provide contact to the active circuitry 2201 such that electrical signals can be transmitted to the active circuitry 2201. In some embodiments, the thickness of the semiconductor wafer 220 may be between about 1 μm and about 10 μm after being thinned down. In some embodiments, the thickness of the semiconductor wafer 220 may be between about 1 μm and about 8 μm, between about 1 μm and about 9 μm, between about 2 μm and about 8 μm, between about 2 μm and about 7 μm, or between about 2 μm and about 6 μm after being thinned down. In some embodiments, a plurality of fluidic channels 2217 extend through the ASIC layer 103. Each fluidic channel 2217 can connect and provide fluidic communication between a middle chamber 2225 in the nanopore layer 101 and a trans chamber 2213 in the post array layer 105.

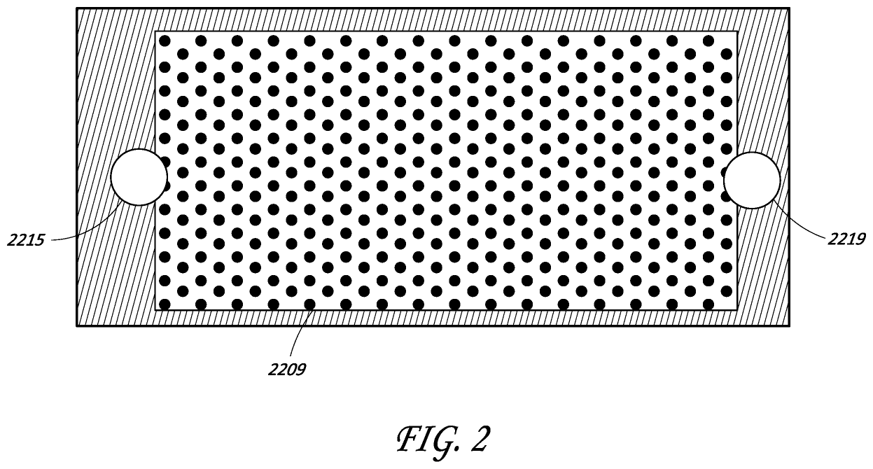

The post array layer 105 includes a support substrate 240 having an array of posts on the top (i.e., post array). The top of the post array 2209 at the front side of the support substrate is bonded to the back side of the semiconductor wafer 220, which creates interconnected gaps. The interconnected gaps form the trans chamber 2213 to serve as fluidic reservoir (e.g., for holding a fluid). Thus, the trans chamber 2213 contains the array of posts 2209 (see FIG. 2). In some embodiments, the post array layer 105 may provide mechanical support to the ASIC layer 103.

The nanopore layer 101 includes a plurality of middle chambers 2225, wherein each of the middle chambers 2225 is positioned over an active circuitry unit 2201, and a membrane 2227 with inserted nanopores 2229 is over the middle chambers 2225. The nanopore 2229 is positioned over each middle chamber 2225, providing fluidic communication between the middle chamber 2225 and a cis chamber. In some embodiments, a fluidic channel 2217 connects each of the middle chambers 2225 to the trans chamber 2213. In some embodiments, the middle chambers 2225 are etched into a polymer layer, and thus each middle chamber is separated from another middle chamber by a polymeric material.

FIG. 1B illustrates a cross-sectional view of a nanopore device 100 according to another embodiment. With reference to FIG. 1B, in some embodiments, the fluidic channel 2217 may further include a horizontal channel 2221 in addition to a vertical section of fluidic channel 2217 that extends through the ASIC layer 103. In some embodiments, the horizontal channel 2221 may take a tortuous/serpentine/meandering path. In some embodiments, the nanopore device 100 may further include an inlet 2215 and an outlet 2219 that allow a fluid, such as an electrolyte, to enter and exit the trans chamber 2213. In some embodiments, the inlet 2215 and outlet 2219 go through the nanopore layer 101 and the ASIC layer 103 to reach the trans chamber 2213 at the front side of the support substrate 240.

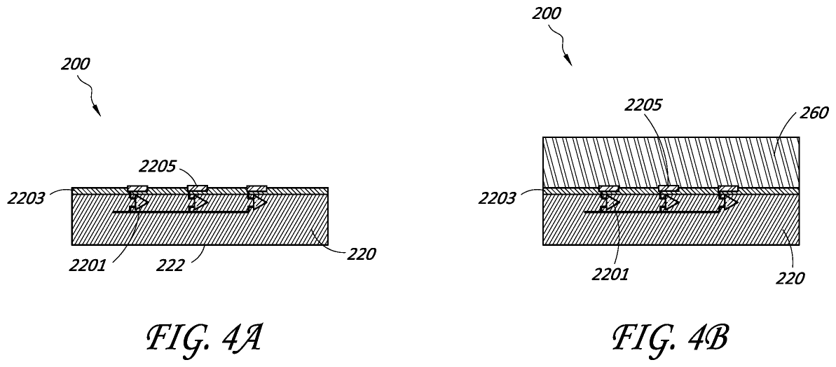

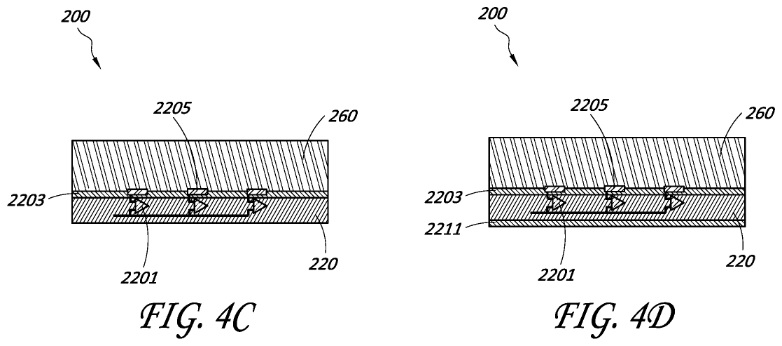

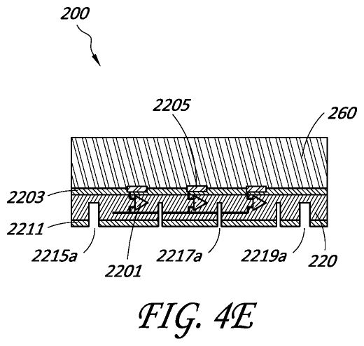

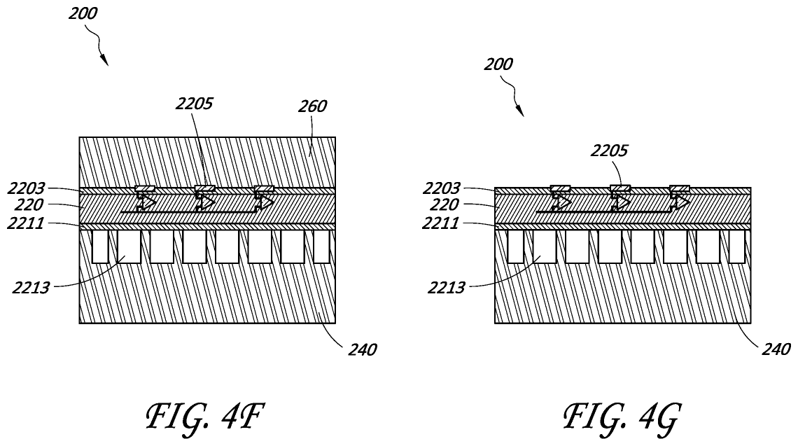

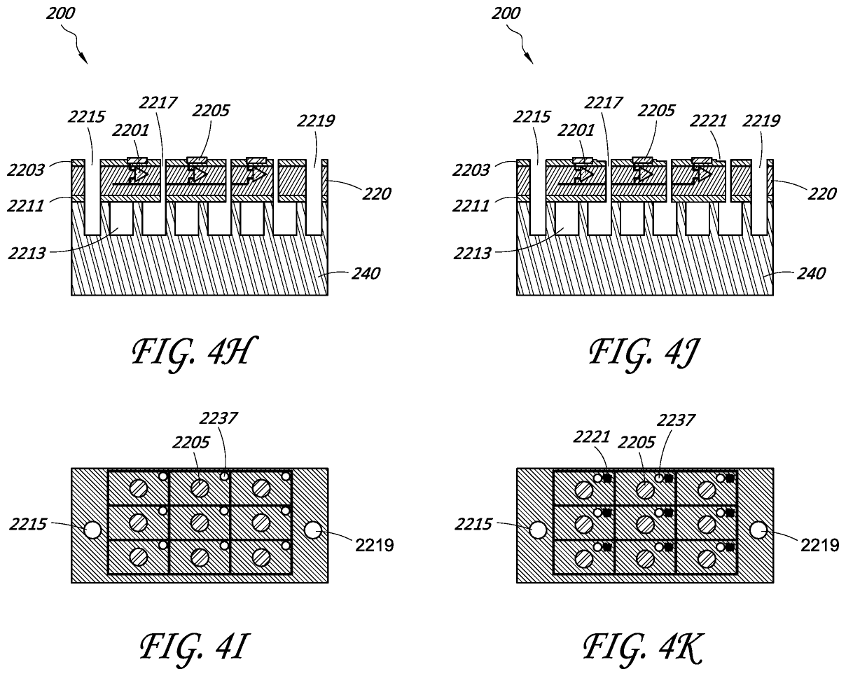

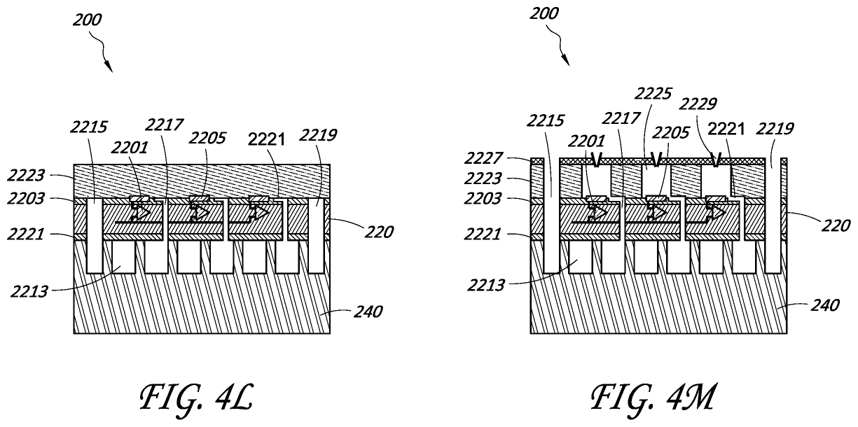

In some embodiments, a first passivation layer 2203 may be disposed on the front side of the ASIC layer 103. In some embodiments, a second passivation layer 2211 may be disposed on the back side of the ASIC layer 103 (see FIG. 4M as an example) after thinning down the semiconductor wafer 220. Both passivation layers 2203 and 2211 may be a dielectric layer.

In some embodiments, such as the embodiment shown in FIG. 1B, a lid 2234 is positioned over the nanopore layer 101 and defines the cis chamber 2235 between the lid 2234 and the membrane 2227. In some embodiments, the inlet 2215 and the outlet 2219 also go through the lid 2234, which allows the fluid to enter and exit the trans chamber 2213 from the inlet and outlet openings in the lid 2234. In some embodiments, two O-rings 2233a and 2233b are placed between the lid 2234 and the membrane 2227 and each surrounds the inlet 2215 and the outlet 2219. The O-rings 2233a and 2233b isolate the inlet 2215 and outlet 2219 from the cis chamber 2235. In some embodiments, the lid 2234 is supported by the O-rings over the nanopore layer 101. In some embodiments, the lid 2234 comprises a transparent or semi-transparent material, such as a polymer, glass, sapphire, or other transparent or semi-transparent materials. In some aspects, the lid 2234 comprising a transparent or semi-transparent material allows for optical inspection of the nanopore 2229 and/or membrane 2227, such as quality of membrane formation, insertion of a protein nanopore into the membrane, and other quality metrics.

FIG. 2 is a top view showing a post array 2209 on the support substrate 240 as shown in FIG. 1A and FIG. 1B. As shown in FIG. 2, the post array 2209 is formed on the front side of the support substrate 240 and span across the front side of the support substrate 240 in two-dimensions. As such, the post array 2209 is a two-dimensional (2D) post array. As shown in FIG. 1A and FIG. 1B, when the post array 2209 contacts with the back side of the semiconductor wafer 220, the post array 2209 may provide mechanical support to the semiconductor wafer 220.

### Process of Making Nanopore System

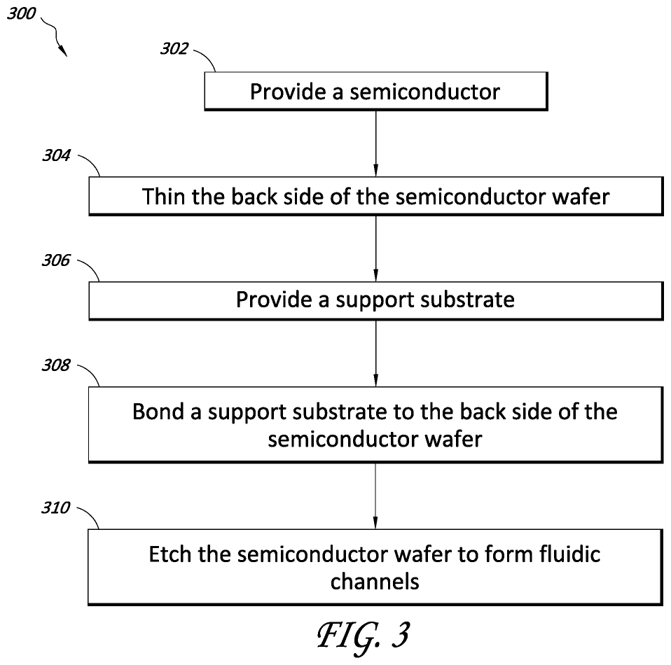

Disclosed herein is a method of forming a bonded structure, such as a nanopore system disclosed herein. In some embodiments, the various layers of the bonded structure are formed separately. FIG. 3 is a flow diagram depicting a method 300 for forming a bonded structure. Block 302 includes providing an ASIC semiconductor wafer, wherein a front side of the semiconductor wafer includes active circuitry. The active circuitry may be built into the front side of the semiconductor wafer. At block 304, the back side of the semiconductor wafer is ground or thinned down. In some embodiments the grinding/thinning step reduces the thickness of the semiconductor wafer to between about 1 μm to about 10 μm. In some embodiments, the thickness of the semiconductor wafer is reduced to about 1 μm and about 8 μm, between about 1 μm and about 9 μm, between about 2 μm and about 8 μm, between about 2 μm and about 7 μm, or between about 2 μm and about 6 μm after thinning. At block 306, a support substrate with a post array on the front side of the support substrate is provided. The post array may be a two-dimensional (2D) post array. Then, at block 308, the support substrate is bonded to the back side of the semiconductor wafer, so the front side of the support substrate is facing the back side of the semiconductor wafer. Step 310 includes forming a plurality of fluidic channels that extend from the front side of the semiconductor wafer through the back side of the semiconductor wafer, and to the front side of the support substrate.

Alternatively, a carrier substrate may be bonded to a front side of the semiconductor wafer before the back side of the semiconductor wafer is thinned at block 304. One advantage of bonding the carrier substrate may be to prevent the semiconductor wafer from being fractured during thinning. With the carrier substrate bonded to the front side of the semiconductor wafer, the semiconductor wafer is less likely to be fractured when thinned down to below 10 μm in thickness.

In some embodiments, the bonding between the carrier substrate and the semiconductor wafer may be temporary. More specifically, the carrier substrate may be de-bonded from the semiconductor wafer after the support substrate is bonded to the back side of the semiconductor wafer at block 308.

In another embodiment, a front side of the support substrate contains a two-post array and the post array makes contact with the back side of the semiconductor wafer at block 308 when the support substrate is bonded to the back side of the semiconductor wafer. An example illustrating the support substrate containing the post array can be found in FIG. 2. The contacts between the post array and the back side of the semiconductor wafer provides mechanical support for the semiconductor wafer. Advantageously, the bonded structure can be more robust with such mechanical support.

In still another embodiment, although not shown in FIG. 3, the method 300 may further include depositing a polymer layer on the front side of the semiconductor wafer, and patterning the polymer layer to form a plurality of fluidic chambers after block 310. The fluidic chambers may be the middle chambers in the nanopore sensor system.

FIGS. 4A-4C demonstrate an example of the process for making the ACIS layer 103 for fabricating the nanopore device shown in FIGS. 1A and 1B. FIG. 4A is a cross-sectional view illustrating a semiconductor wafer 220 (e.g., a silicon wafer) having active circuitry 2201 built into the front side according to one embodiment. The active circuitry 2201 may be application specific integrated circuits (ASIC) that include two-dimensional (2D) active electronic measurement elements. In some embodiments, after the active circuitry 2201 is built in the semiconductor wafer 220, the surface of the front side of the semiconductor wafer 220 may be passivated by disposing a dielectric layer 2203 (e.g., SiO₂. SiN, SiON) on the surface of the front side. In some embodiments, etching may be performed on the surface of the front side of the semiconductor wafer 220 to expose the conductive pads 2205. The conductive pads 2205 may provide contact to the active circuitry 2201 such that electrical signals can be transmitted to the active circuitry 2201.

FIGS. 4B and 4C illustrate the process of thinning the ASIC layer 103 according to one embodiment. A carrier substrate 260 may be bonded to the front side of the semiconductor wafer 220 containing the active circuitry 2201 prior to grinding or thinning down the semiconductor wafer 220 from the back side of the semiconductor wafer 220. In some embodiments, the carrier substrate may comprise sapphire, glass, dielectric, polymer, metal, silicon or a semiconductor. In some embodiments, thinning of the semiconductor wafer 220 can be carried out by chemical-mechanical polishing (CMP) or other comparable processes. Before thinning, the semiconductor wafer 220 may have a thickness of above about 700 μm. Alternatively, thinning of the semiconductor wafer 220 may be carried out without bonding the carrier substrate 260 to the front side of the semiconductor wafer 220 (not shown). However, the bonding the carrier substrate 260 to the semiconductor wafer 220 before the grinding or thinning may provide an advantage such as reducing the likelihood of semiconductor wafer 220 being fractured during the thinning process.

FIG. 4C is a cross-sectional view illustrating the thinned down semiconductor wafer 220 according to one embodiment. In contrast to FIG. 4B, the back side of the semiconductor wafer 220 has been thinned down. After thinning, the semiconductor wafer 220 may be about 1 to 10 μm in thickness. As discussed above, with the thickness of the semiconductor wafer 220 being around or less than 10 μm, short TSVs can be adopted to form the fluidic channels 2217 in subsequent stages and both the cost and time for etching may be reduced.

FIG. 4D is a cross-sectional view illustrating an optional step of depositing a second passivation layer 2211 (such as a dielectric layer) to the back side of the semiconductor wafer 220 according to one embodiment. As shown, the second passivation layer 2211 is deposited on the back side of the semiconductor wafer 220 after the back side of the semiconductor wafer 220 is thinned. The second passivation layer 2211 may be a dielectric layer. The second passivation layer 2211 may include SiO₂, silicon nitroxides, Si₃N₄, or other dielectric materials.

FIG. 4F is a cross-sectional view illustrating the bonding of a support substrate 240 to the semiconductor wafer 220 according to one embodiment. As shown, the front side of the support substrate 240 containing a post array is bonded to the back side of the semiconductor wafer 220. After the bonding, the top of the post array 2209 (as shown in FIG. 2) is in contact with the back side of the semiconductor wafer 220 to from the trans chamber 2213. In some embodiments, the semiconductor wafer 220 may be mechanically supported by the 2D post array 2209. In some embodiments, a metal layer may be deposited on the bottom of the trans chamber 2213 before bonding the support substrate 240 to the semiconductor wafer 220, which may enhance electric connectivity.

In some embodiments, a dielectric layer may be disposed on the front side of the support substrate 240, and dielectric layer may be etched to form the post array 2209. In some embodiments, the dielectric layer may comprise SiO₂, silicon nitroxides, Si₃N₄, or other dielectric materials. The dielectric layer may be deposited or formed by one or more processes such as chemical vapor deposition (CVD), physical vapor deposition (PVD), or rapid thermal processing (RTP). The support substrate 240 may be a semiconductor substrate, sapphire, glass, dielectric, polymer or other suitable types of substrates.

Once the support substrate 240 with the post array 2209 is bonded to the back side of the semiconductor wafer 220, the carrier substrate 260 may be de-bonded (e.g., removed) from the semiconductor wafer 220 as shown in FIG. 4G. As such, the bonding of the semiconductor wafer 220 and the carrier substrate 260 may be temporary. After de-bonding, the front side of the semiconductor wafer 220 is revealed.

The next step in the process involves forming a plurality of fluidic channels. In some embodiments, an inlet for introducing a fluid to the trans chamber and an outlet for the fluid to flow out of the trans chamber are also formed. With reference to FIG. 4H, an inlet 2215, an outlet 2219 and a plurality of fluidic channels 2217 may be formed by etching from the front side of the semiconductor wafer 220 using plasma etching, wet etching or combinations of both plasma and wet etching. As shown, the fluidic channels 2217 extend from the front side of the semiconductor wafer 220 to the back side of the semiconductor wafer 220 to reach the front side of the support substrate 240.

In some embodiments, the fluidic channels 2217, and optionally inlet 2215 and outlet 2219, may be partially etched from the backside of the semiconductor wafer 220 before bonding the support substrate 240 with post array 2209 to the semiconductor wafer 220. With reference to FIG. 4E, the fluidic channels 2217a and optionally inlet 2215a and outlet 2219a may be etched halfway or close to halfway into the semiconductor wafer 220 from the back side. Advantageously, partial or halfway etching illustrated in FIG. 4E may make the fluidic channels 2217 in FIG. 4H easier to etch through from the front side of the semiconductor wafer 220 and the diameter may be further reduced. Alternatively, the fluidic channels 2217, and optionally inlet 2215 and outlet 2219, can then be completed etched through the semiconductor wafer 220 from the front side of the semiconductor wafer 220 after the support substrate 240 is bonded to the semiconductor wafer 220 (and after debonding the carrier substrate 260 in some embodiments).

As shown in FIG. 4I, which is the top view of the ASIC layer, the fluidic channels 2217 have openings 2237 on the first passivation layer 2203 (or on the semiconductor wafer 220 if the passivation layer is not present). Each opening 2237 is positioned nearby each conductive pad 2205. With the semiconductor wafer 220 thinned down to a thickness of around or less than about 10 μm, the cost and time of etching the fluidic channels 2217, the inlet 2215, and the outlet 2219 can be reduced markedly. Further, the diameters of the fluidic channels 2217 can be made smaller (e.g., below about 30 μm). As such, more fluidic channels may be formed in a flow cell, thus increasing the density of the nanopore sensing units.

FIGS. 4J and 4K are cross-sectional and top views illustrating the partially assembled flow cell according to one embodiment. In some embodiments, horizontal channels 2221 may be further etched on the passivation layer 2203 (or the semiconductor wafer 220 if the passivation layer is not present). Plasma etching, wet etching or combinations of both plasma and wet etching can be utilized to etch the horizontal channels 2221. As shown in FIG. 4K, the horizontal channels 2221 may be tortuous in shape to provide desired resistance. Advantageously, by connecting the resistance of the horizontal channels 2221 and the fluidic channels 2217 in series, desirable resistance for serving as voltage divider can be provided.

Alternatively, the horizontal channels 2221 may not be needed and the fluidic channels 2217 can still provide enough resistance. By way of adopting short TSVs because of a thickness of the semiconductor wafer 220 being around or less than 10 μm, the fluidic channels 2217 may be made sufficiently thin in diameter (e.g., less than 30 μm). As such, the resistance value of the fluidic channels 2217 alone may be enough for the voltage divider application.

Next, the nanopore layer may be formed on top of the ASIC layer. With reference to FIGS. 4L and 4M, a polymer layer 2223 may be laminated on the front side of the semiconductor wafer 220. The polymer layer 2223 may be SU-8 photoresist or other dry film photoresists. The middle chambers 2225 may be formed within the polymer layer 2223 by plasma etching, wet etching or combinations of both plasma and wet etching. The middle chambers 2225 are positioned over each of the conductive pads 2205 and the openings of the fluidic channels. The membrane 2227 may be disposed on top of the polymer layer 2223. A nanopore 2229 can then be inserted through the membrane 2227 over each middle chamber 2225. In some embodiments of the nanopore device, one of the middle chambers 2225 may be associated with one of the nanopores 2229, one of the conductive pads 2205, one of the fluidic channels 2217 and one of the active electronic measurement elements in the active circuitry 2201 to form a nanopore sensor unit as described above. One of the active electronic measurement elements may be used to detect the voltage variation associated with the voltage divider formed by one of the fluidic channels 2217 and one of the nanopores 2229 as the molecule to be identified passes through the nanopores 2229.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 nm to about 20 nm should be interpreted to include not only the explicitly recited limits of from about 2 nm to about 20 nm, but also to include individual values, such as about 3.5 nm, about 8 nm, about 18.2 nm, etc., and sub-ranges, such as from about 5 nm to about 10 nm, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A nanopore device, comprising:
an application specific integrated circuit (ASIC) layer, wherein the ASIC layer comprises active circuitry on a front side of a thinned-down semiconductor wafer;
a post array layer under the ASIC layer, wherein the post array layer comprises an array of posts on a front side of a support substrate; and
a nanopore layer over the ASIC layer, wherein the nanopore layer comprises a plurality of middle chambers over the active circuitry, a membrane on the plurality of middle chambers, and a plurality of nanopores inserted in the membrane over the plurality of middle chambers.

2. The nanopore device as defined in claim 1, further comprising:
a cis chamber over the nanopore layer, wherein at least one of the plurality of nanopores fluidically connects the cis chamber to at least one of the plurality of middle chambers; and
a trans chamber under the ASIC layer, wherein the trans chamber comprises the array of posts.

3. The nanopore device as defined in claim 2, further comprising a plurality of fluidic channels extending through the ASIC layer, wherein at least one of the plurality of fluidic channels fluidically connects at least one of the plurality of middle chambers to the trans chamber.

4. The nanopore device as defined in claim 3, wherein at least one of the plurality of fluidic channels comprises a horizontal channel and a vertical channel.

5. The nanopore device as defined in claim 3, further comprises a fluid inlet and a fluid outlet reaching the trans chamber through the nanopore layer and the ASIC layer.

6. The nanopore device as defined in claim 1, wherein the ASIC layer has a thickness of about 1 μm to about 10 μm.

7. The nanopore device as defined in claim 1, wherein the support substrate comprises silicon, semiconductor, sapphire, dielectric, polymer or glass.

8. A method of forming a bonded structure, the method comprising:
providing a semiconductor wafer, wherein a front side of the semiconductor wafer comprises active circuitry;
thinning a back side of the semiconductor wafer;
providing a support substrate comprising a post array on a front side of the support substrate;
bonding the support substrate to the back side of the semiconductor wafer after said thinning the back side of the semiconductor wafer such that the front side of the support substrate is in contact with the back side of the semiconductor wafer; and
forming a plurality of fluidic channels that extend from the front side of the semiconductor wafer to the front side of the support substrate.

9. The method as defined in claim 8, further comprising bonding a carrier substrate to the front side of the semiconductor wafer before said thinning the back side of the semiconductor wafer.

10. The method as defined in claim 9, wherein a thickness of the semiconductor wafer is between about 1 μm and about 10 μm after said thinning the back side of the semiconductor wafer.

11. The method as defined in claim 9, further comprising debonding the carrier substrate from the front side of the semiconductor wafer after said thinning the back side of the semiconductor wafer.

12. The method as defined in claim 9, wherein the carrier substrate is made of sapphire, glass, dielectric, polymer, metal, silicon or semiconductor.

13. The method as defined in claim 8, wherein the post array is in contact with the back side of the semiconductor wafer.

14. The method as defined in claim 8, further comprising partially forming the plurality of fluidic channels in the semiconductor wafer extending from the back side of the semiconductor wafer.

15. The method as defined in claim 8, further comprising depositing a polymer layer on the front side of the semiconductor wafer; and patterning the polymer layer to form a plurality of fluidic chambers, wherein at least one of the plurality of fluidic chambers fluidically connects with at least one of the plurality of fluidic channels.

16. The method as defined in claim 15, further comprising disposing a membrane comprising a plurality of nanopores on the polymer layer, wherein the plurality of nanopores provide fluid access to the plurality of fluidic chambers.

17. The method as defined in claim 16, further comprising forming a fluidic inlet port and a fluidic outlet port through the membrane, the polymer layer, and the semiconductor wafer to reach the front side of the support substrate.

18. The method as defined in claim 8, wherein providing the support substrate comprises etching the front side of the support substrate to form the post array.

19. The method as defined in claim 8, wherein providing the support substrate further comprises depositing a metal layer on a bottom of the post array.

20. The method as defined in claim 8, wherein the support substrate comprises silicon, semiconductor, sapphire, dielectric, polymer or glass.

21. The method as defined in claim 8, wherein the support substrate further comprises a dielectric layer on the front side of the support substrate.

22. The method as defined in claim 8, wherein providing the support substrate comprises depositing a dielectric layer on a substrate and etching the dielectric layer to form the post array on the front side of the support substrate.
